(19) United States
(12) Patent Application Publication
(10) Pub. No.: US 2026/0183448 A1
(43) Pub. Date: Jul. 2, 2026

(54) BENDABLE TITANIUM-ALLOY IMPLANTS, AND RELATED SYSTEMS AND METHODS

(71) Applicant: DePuy Ireland Unlimited Company, Cork (IE)

(72) Inventors: Edward Patrick Kavanagh, Cork (IE); David Horne, Leeds (GB); Charlie Weston, West Yorkshire (GB); Ian Flatters, Sheffield (GB); João Arranhado Neves, Cork (IE); Fionnán Aodhán McNamara, County Carlow (IE); Enda Leavy, Cork (IE); Karen McKinlay, Everton (GB); Nazir Ahmed Karbanee, York (GB); Philippa Clarkson, Leeds (GB)

(21) Appl. No.: 19/546,549

(22) Filed: Feb. 23, 2026

Related U.S. Application Data
(63) Continuation of application No. 17/732,862, filed on Apr. 29, 2022.

(51) Int. Cl. A61L 27/06; A61F 2/34; A61L 27/50

(52) U.S. Cl. CPC A61L 27/06; A61F 2/34; A61L 27/50; A61F 2002/342; A61F 2002/343; A61F 2002/3432; A61F 2310/00023; A61L 2430/24

(57) ABSTRACT

An implantable device includes a portion that is constructed such that at least a majority of the portion, as measured by volume, comprises Ti64(Ti-6Al-4V) alloy. Additionally, the portion is bendable to a bend angle of at least about 50-degrees about a bend axis while maintaining structural integrity.

### CROSS-REFERENCE TO RELATED APPLICATIONS

This is a continuation of now-allowed U.S. patent application Ser. No. 17/732,862, filed Apr. 29, 2022. All foregoing applications are incorporated herein by reference in their entireties for any and all purposes.

### TECHNICAL FIELD

The present invention relates to titanium-alloy implants, and more particularly to processes for rapidly manufacturing such implants and providing such implants with increased malleability.

### BACKGROUND

The field of rapid prototyping and additive manufacturing has seen many advances over the years, particularly for rapid prototyping of articles such as prototype parts and mold dies. These advances have reduced fabrication cost and time, while increasing accuracy of the finished product, versus conventional machining processes, such as those where materials (e.g., metal) start as a block of material, and are consequently machined down to the finished product.

However, the main focus of rapid prototyping three-dimensional structures, such as surgical implants, has been on increasing density of rapid prototyped structures. Examples of modern rapid prototyping/additive manufacturing techniques include sheet lamination, adhesion bonding, laser sintering (or selective laser sintering), laser melting (or selective laser melting), photopolymerization, droplet deposition, stereolithography, 3D printing, fused deposition modeling, and 3D plotting. Particularly in the areas of selective laser sintering, selective laser melting and 3D printing, the improvement in the production of high-density parts has made those techniques useful in designing and accurately producing articles such as highly dense metal parts. Those techniques, however, often require post-formation treatment and/or finishing processes to provide the prototyped part with the mechanical properties (e.g., hardness, strength, malleability, toughness) necessary to meet the demands required for various types of surgical implants.

### SUMMARY

According to an embodiment of the present disclosure, an implantable device includes a portion that is constructed such that at least a majority of the portion, as measured by volume, comprises Ti64 (Ti-6Al-4V) alloy. Additionally, the portion is bendable to a bend angle of at least about 50-degrees about a bend axis while maintaining structural integrity.

According to another embodiment of the present disclosure, an acetabular cage has a dome for insertion within an acetabulum and a flange that is monolithic with the dome and is for affixation to a portion of an ilium. The dome and the flange are constructed of Ti64 alloy. At least a portion of the flange is bendable to a bend angle of at least about 90 degrees about a bend axis while maintaining structural integrity.

According to an additional embodiment of the present disclosure, a method of preparing an implant for implantation includes bending a portion of the implant to a bend angle of at least about 50-degrees about a bend axis. At least a majority of the portion as measured by volume comprises Ti64 alloy. The portion maintains structural integrity during and after the bending step.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1A is a perspective view of a surgical implant attached to patient anatomy, according to an embodiment of the present disclosure;

FIG. 1B is a perspective view of the implant attached to a complimentary second implant and to patient anatomy, according to an embodiment of the present disclosure;

FIG. 1C is a perspective view of the implant illustrated in FIG. 1A;

FIG. 1D is another perspective view of the implant illustrated in FIG. 1A;

FIGS. 1E and 1F are perspective views of additional embodiments of a surgical implant similar to the implant shown in FIG. 1A, each according to an embodiment of the present disclosure;

FIG. 2A is a perspective view of a partially manufactured version of the implant illustrated in FIG. 1A, shown at an example build orientation with respect to an additive manufacturing powder bed;

FIG. 2B is a perspective view of a partially manufactured version of the implant illustrated in FIG. 1A, shown at an alternative build orientation with respect to the powder bed;

FIGS. 3A and 3B show magnified views of exemplary grain structures of a Ti64 alloy having increased malleability for use in implant construction; the grain structures shown in FIGS. 3A and 3B were produced in the same Ti64 material using slightly different custom heat treatment processes, which allow the size of the alpha-phase lamella to be tailored; for example, the average width of the alpha-phase lamella in FIG. 3B are larger than those of FIG. 3A;

FIGS. 3C and 3D show additional magnified views of grain structure of the Ti64 alloy produced using the same custom heat treatment process used to produce the grain structure shown in FIG. 3B;

FIG. 3E shows a magnified view of a grain structure of a prior art sample of ISO 20160 alpha+beta titanium alloy;

FIGS. 4A and 4B are charts showing tensile test results of structural test members having grain structures that are substantially similar to the grain structure illustrated in FIGS. 3A-3B;

FIG. 5A is a chart comparing bend test data of structural test members having grain structures that are substantially similar to the grain structure illustrated in FIGS. 3A-3B with structural test members lacking the aforementioned grain structure;

FIG. 5B is a photograph of the resulting bent structural test members having grain structures that are substantially similar to the grain structure illustrated in FIGS. 3A-3B, following the bending tests used to generate the results illustrated in FIG. 5A;

FIG. 5C is a photograph of the resulting bent structure test members that lack the grain structure illustrated in FIGS. 3A-3B, following the bending tests used to generate the results illustrated in FIG. 5A;

FIG. 5D is a chart comparing additional bend test data of structural test members having grain structures that are substantially similar to the grain structure illustrated in FIGS. 3A-3B with structural test members lacking the aforementioned grain structure; and

FIG. 6 is a chart showing yet additional bend test data of structural test members having various thicknesses and having grain structures that are substantially similar to the grain structure illustrated in FIGS. 3A-3B; and

FIG. 7 is an exploded, perspective view of a surgical system having a bendable implant and a bendable, hardenable trial member, according to an embodiment of the present disclosure.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “titanium-based”, as used herein with respect to an object, such as an implant, means constructed predominantly of titanium (Ti) or a titanium alloy.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although the terms first, second, etc. may be used herein to describe various elements, these elements should not be limited by these terms. These terms are instead used to distinguish one element from another. For example, a first element could be termed a second element, and, similarly, a second element could be termed a first element without departing from the scope of the embodiments disclosed herein.

The embodiments disclosed herein pertain to titanium-alloy surgical implants having superior strength and also increased malleability, which allows the implants to be bent intra-operatively, such as for shaping to correspond to associated patient anatomy. The surgical implants disclosed herein are also suitable for rapid manufacturing via additive manufacturing processes and subsequent heat treatment processes configured to provide the manufactured implant with increased malleability. Titanium (Ti) and various titanium alloys, particularly Ti64 (a titanium-aluminum-vanadium alloy, also denoted as Ti-6Al-4V), have mechanical properties that make them desirable for surgical implants, including strength, lighter weights (lower density) compared to other implant materials (e.g., stainless steel), durability, resistance to repeated loads, the ability to withstand strain (e.g., during internal fixation to patient anatomy), corrosion resistance, biocompatibility, and the ability to bond with bone. Although titanium-alloy implants possess many of these superior properties, they tend to be difficult to bend, particularly during intra-operative shaping processes for matching the implant shape to patient anatomy. The titanium-alloy implants disclosed herein, however, have grain structures that provide the implants with increased malleability, which allows for intra-operative bending, such as for shaping the implants to associated patient anatomy. Non-limiting examples of types of surgical implants that benefits from the malleable titanium-alloy discloses herein includes acetabular cages, craniomaxillofacial (CMF) plates, and trauma bone plates. Although the following illustrated embodiments of the manufacturing, treatment, and finishing processes and methods of implant use refer to acetabular cages, it should be appreciated that the processes, methods, and structures disclosed herein can be employed for the construction and use of numerous other types of implants.

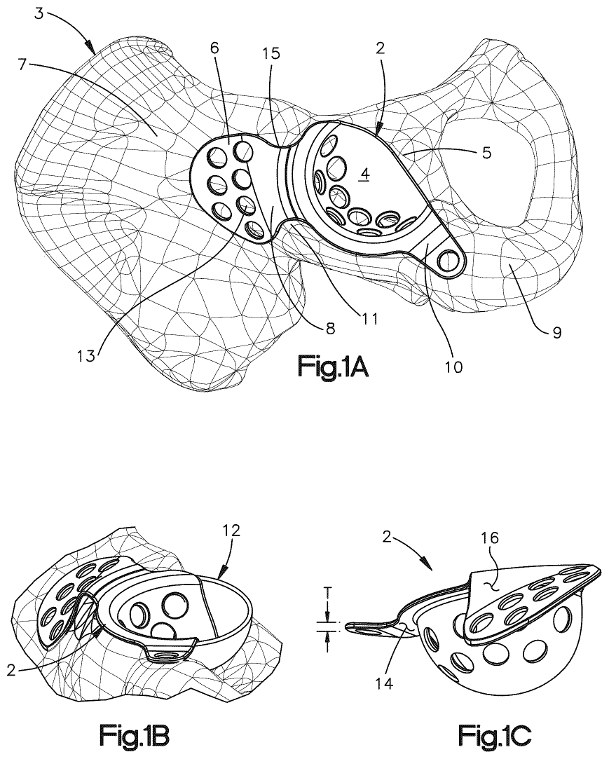

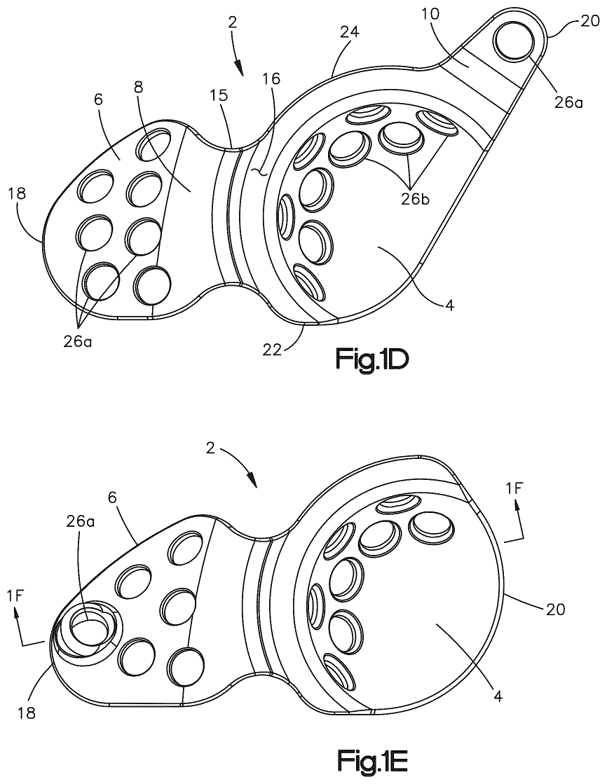

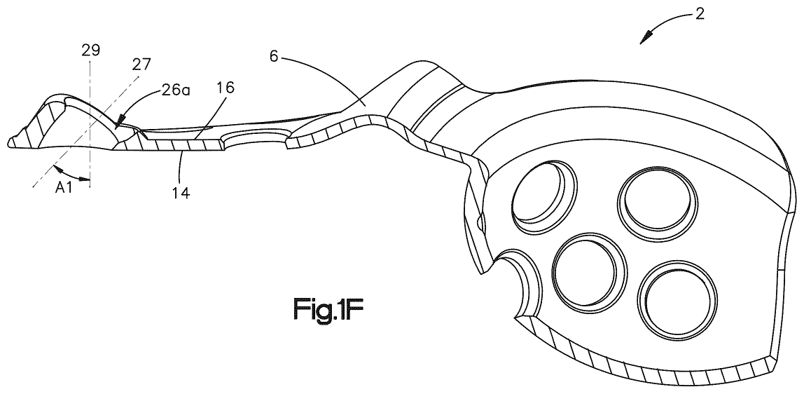

Referring to FIGS. 1A-1C, an exemplary implant 2 for hip repair is shown. The illustrated implant 2 is referred to herein as an “acetabular cage” 2 and is configured for affixation to the acetabulum 5 for repair of various acetabular defects in the pelvis 3. The cage 2 includes a socket portion or “dome” 4 shaped for insertion and affixation within the acetabulum 5. The cage 2 also includes a first flange 6 that extends from the dome 4 and is configured for affixation to the ilium 7 of the pelvis 3. The first flange 6 is also referred to herein as the “iliac flange” 6. The iliac flange 6 also defines a bend 8, particularly an S-type bend (“S-bend”) 8, at a portion thereof proximate the dome 4. The S-bend 8 is configured to accommodate and generally conform to the geometry of the acetabular margin (rim) 11 and the body of ilium 13. The iliac flange 6 can also define a neck 15 adjacent the dome 4. The neck 15 is configured for facilitating engagement of the iliac flange 6 with the acetabular margin 11 and also for facilitating bending and/or twisting of the iliac flange 6 to match patient anatomy. The cage 2 can also include an optional second flange 10 that extends from the dome 4 at a location generally opposite the iliac flange 6 and is configured for affixation to the ischium 9 of the pelvis 3. The second flange 10 is also referred to herein as the “ischial flange” 10. In other embodiments, as shown in FIG. 1E, the cage 2 can be formed having the iliac flange 6 but lacking an ischial flange.

It should be appreciated that the dome 4 can be configured to seat within the acetabulum 5 and contact the acetabular surface(s) (e.g., the articulate (lunate) surface, the acetabular fossa, and/or reamed or otherwise surgically modified portions thereof), as shown in FIG. 1A, and can also be configured to seat within an acetabular cup 12 that is, in turn, in direct contact with the acetabular surfaces, as shown in FIG. 1B. The cage 2 has sufficient malleability to allow a surgeon to manipulate, bend, and shape portions thereof so as to conform to the shape of adjacent patient anatomy. In particular, the iliac flange 6 and the optional ischial flange 10 can be bent and shaped to have contours that match the associated contours of the ilium 7 and ischium 9, respectively. One or more portions of the cage 2 can have geometric features that further facilitate bending. For example, the neck 15 of the iliac flange 6 is configured to facilitate bending, twisting, and shaping of the iliac flange 6.

Referring now to FIGS. 1C-1D, the cage 2 has a first or inner surface 14 that is configured to face the pelvis when implanted and an opposed second or outer surface 16 that is configured to face away from the pelvis 3 when implanted. The cage 2 defines a thickness T measured between corresponding portions of the inner and outer surfaces 14, 16 along a direction orthogonal thereto. The cage thickness T is preferably in a range of about 0.5 mm to about 3.0 mm, and more particularly in a range of about 0.9 mm to about 2.2 mm, and more particularly in a range of about 0.9 mm to about 1.5 mm. It should be appreciated that one or more portions of the cage 2 can have a reduced thickness relative to various other portions of the cage 2. In such embodiments, the portion(s) having reduced thickness can be configured to facilitate bending and shaping along such portion(s).

The cage 2 has a first or superior end 18, which is located at the end of the iliac flange 6, and a second or inferior end 20, which, in the embodiments having the ischial flange 10, is located at the end of the ischial flange 10. In embodiments lacking an ischial flange, the inferior end 20 of the cage 2 can be at the inferior-most edge of the dome 4. The cage 2 also has an anterior side 22 and an opposed posterior side 24. It should be appreciated that anatomical directional terms, such as “medial”, “lateral”, “superior”, “inferior”, “anterior”, and “posterior”, when used herein with reference to an implant, such as the acetabular cage 2, refer generally to the relative positions of the implant as it is configured to be oriented when implanted.

The cage 2 includes fixation features for affixation to bone. As shown in FIGS. 1D-1E, the dome 4, the iliac flange 6, and the optional ischial flange 10 can each include one or more respective fixation holes 26a,b for receiving respective fixation members, such as bone screws, for affixing the associated portion of the cage 2 to underlying bone. For example, the iliac flange 6 and/or the optional ischial flange 10 can include one or more flange holes 26a for affixing the respective flange 6, 10 to underlying bone. One or more of the flange holes 26a can also have interior threads for locking with complimentary threads on the exterior of a head of a locking screw.

As shown in FIG. 1F, one or more of the flange holes 26a can extend through the respective flange 6, 10 along a central hole axis 27 that is oriented at an angle A1 offset from a transverse axis 29 extending orthogonally through the inner and outer surfaces 14, 16 of the cage 2 adjacent the hole 26a. Such a hole 26a can be referred to as an “angled hole.” It should also be appreciated that one or more of the flange holes 26a, including angled and non-angled holes 26a, can have threads or other locking structures for engaging, in locking fashion, complimentary threads on the head of a variable-angle (VA) locking screw, thereby providing VA fixation therewith. It should be appreciated that the inclusion of at least one angled flange hole 26a can provide significant advantages for the cage 2. One such advantage is that the angle A1 of the hole 26a can be tailored to provide a targeted insertion trajectory for the associated bone screw. In this manner, the angle A1 can be adapted for tailoring the insertion trajectory to the particular geometry of the target portion of bone, which can provide enhanced fixation with the target portion of bone. For example, the angle A1 of a hole 26a on the iliac flange 6 can be adapted to provide an insertion trajectory that is tailored for the particular geometry of the target portion of ilium, such as an insertion trajectory that is substantially parallel with the bone stock at the target ilium portion. In this manner, the angle A1 can be adapted to increase the efficiency at which the bone screw bites into or otherwise engages the target bone. Another advantage of angled holes 26a is that the angle A1 can be adapted to reduce the amount of soft tissue disruption (e.g., incision, resection, and/or retraction) adjacent the hole 26a. For example, the angle A1 of a hole 26a in the iliac flange 6 can be tailored to reduce the amount of gluteal muscle resection necessary to affix the iliac flange 6 to the associated portion of the ilium.

It should be appreciated that such VA locking hole(s) 26a in the iliac and/or ischial flange 6, 10 can be configured as more fully described in U.S. Pat. No. 10,772,665, issued Sep. 15, 2020, in the name of Bosshard et al. (“the '665 Reference”); and U.S. Pat. No. 11,013,541, issued May 25, 2021, in the name of Bosshard et al. (“the '541 Reference”), the entire disclosures of each of which are hereby incorporated by reference herein. Additionally or alternatively, one or more of the holes 26a in the iliac and/or ischial flange 6, 10 can be a VA locking hole 26a having a polygonal hole shape (as viewed in a reference plane orthogonal to a central axis of the respective hole), such as a trigonal hole shape. For example, the iliac flange 6 and/or the optional ischial flange 10 can include one or more trigonal VA locking flange holes 26a, which can be configured as more fully described in U.S. Pat. No. 11,179,180, issued Nov. 23, 2021, in the name of Oberli et al. (“the '180 Reference”); and U.S. Patent Publication No. 2021/0015526 A1, published Jan. 21, 2021, in the name of Oberli et al. (“the '526 Reference”), the entire disclosures of each of which are hereby incorporated by reference herein. For embodiments of the cage 2 that are configured for use with an acetabular cup 12, the dome 4 can have one or more fixation holes 26b that are preferably configured to align with one or more corresponding fixation holes in the acetabular cup 12.

Select portions of the cage 2 can have grip features for providing a stronger contact interface with the underlying bone. For example, one or more select portions of the inner surface 18 can have ridges thereon for pressing into underlying bone in a manner enhancing fixation. Additionally or alternatively, the cage 2 material along one or more select portions of the inner surface 18 can be porous, i.e., can have a porous three-dimensional (3D) structure that defines a multiplicity of pores, which can be tailored according to various design objectives, such as for promoting boney ingrowth into the pores for enhanced post-surgery fixation with the underlying bone, by way of a non-limiting example. Features of the porous structure, including variations of such features, are described in more detail below.

The implants of the present disclosure, such as the cage 2 of the embodiments illustrated herein, are constructed of a titanium alloy, particularly a titanium-aluminum-vanadium alloy: Ti-6Al-4V (“Ti64”). This particular titanium alloy (Ti64) is advantageous for the construction of implants because Ti64 is stronger and withstands fatigue better than titanium (including grades of “commercially pure” titanium (Ti-CP)) and other titanium alloys. However, prior art implants, such as acetabular cages, that are constructed of Ti64 are difficult to bend, particularly by a physician intra-operatively, and particularly without crack formation at the bend regions. The heat treatment processes discussed below have demonstrated the ability to increase the malleability of implants constructed of Ti64 so that these cages 2 can be bent, e.g., intra-operatively by a physician, without losing structural integrity. In this manner, the inventors have discovered techniques allowing for the strength and durability of Ti64 to be employed into surgical implants (e.g., acetabular cages 2) that are also bendable, which represents a significant advancement in the art of surgical implants.

The use of Ti64 as the implant material also allows the cage 2 to be rapidly manufactured by an additive manufacturing process, such as a laser-activated powder bed fusion (PBF) process, which forms the cage 2 into a desired, pre-operative shape. Features of the implant manufacturing and finishing processes will now be described.

The geometry of the cage 2 can be created in three-dimensional (3D) virtual space, thereby providing a 3D virtual model of the cage 2. After the 3D virtual model of the cage 2 is created, the rapid additive manufacturing processes can be employed to create the physical version of the cage 2 possessing the geometry and features of 3D virtual model. The 3D virtual model of the cage 2 can optionally be designed with the assistance of patient scan data of the specific anatomy to which the cage 2 will be affixed. Such patient scan data can include, by way of a non-limiting example, a series of CT-scan slices of the acetabulum and the adjacent portions of the ilium 7 and ischium 9. In particular, the iliac flange 6 and the optional ischial flange 10 can be tailored in 3D virtual space to have contours that correspond to those of the respective portions of the ilium 7 and ischium 9 to which the flanges 6, 10 will affix. After the 3D virtual model of the cage 2 is created, the rapid additive manufacturing processes can be employed to create the cage 2 possessing the tailored, patient-specific 3D virtual geometry.

The cage 2 can be constructed using a laser-activated PBF process, which can also be referred to as a laser-fusion process, which encompasses selective laser sintering (SLS) and selective laser melting (SLM) processes. A non-limiting example of a laser-fusion process for building a cage 2 will now be described. To begin, a thin layer of powder is dispensed on a working table (frequently referred to as the “build platform”), so that at least one layer of powder forms a powder bed. The powder comprises constituent particles of Ti64 material, which particles can be of various sizes, size distributions, and geometrical shapes, which powder characteristics can be selected based parameters conducive for favorable fusing and fused grain structure of the built cage 2. As used herein, the terms “build”, “built”, and derivates thereof refer to the construct formed by fusing particles within the powder bed together by the PBF process. Selected areas of the top surface of the powder layer (the “build surface”) are fused by exposure to a directed energy source, typically a laser beam. The exposure pattern of the laser beam thus forms a cross-section of the three-dimensional object. The cage 2 is built through consecutive fusion of so-formed cross-sections that are stacked in layer-by-layer fashion along a vertical direction. Between the fusion of each layer, the build platform is incremented downward and a new layer of powder is deposited onto the build surface. These steps (e.g., lowering the working table, distributing a new powder layer atop the powder bed, and exposing the new powder layer to the laser source 1) are repeated, layer-by-layer, as needed, thereby fabricating the cage 2 as a plurality of consecutively fused cross-sectional layers. It should be appreciated that the laser-fusion process can also be characterized as a 3D printing process. Accordingly, the term “built” can be used synonymously herein with the term “printed.”

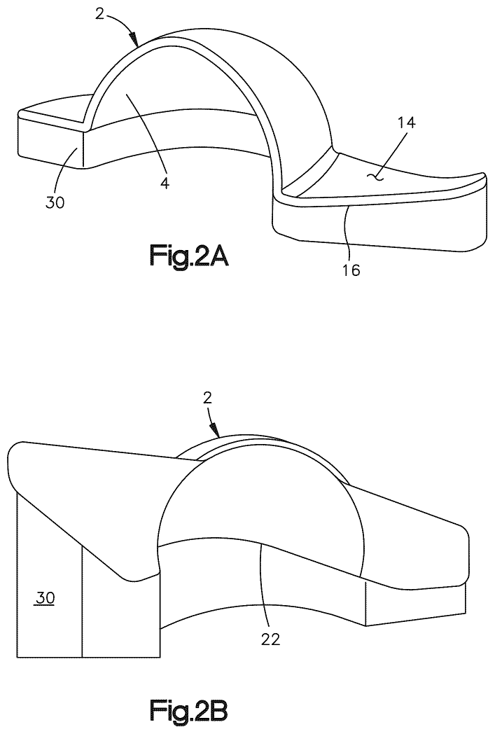

During the PBF process, the cage 2 can be built at various orientations in the power bed. Two non-limiting examples of such build orientation are shown in FIGS. 2A-2B. As shown in FIG. 2A, the cage 2 can be built so that the outer surface 16 faces downward in the powder bed (and so that the inner surface 14 faces upward in the powder bed). Alternatively, as shown in FIG. 2B, the cage 2 can be built so that the anterior side 22 faces downward (and the posterior side 24 faces upward) in the powder bed. At either of these build orientations (and at other build orientations), the cage 2 can be built with a base member 30 of stock material underneath, which base member 30 can be employed as a support structure during post-build processes and subsequently removed. As shown, the cage 2 can be built in the powder bed without fixation holes, which can be subsequently formed in the cage 2 during a machining process, such as by a multi-axis Computer Numerical Control (CNC) machine. In other embodiments, the cage 2 can be built having fixation holes, which can be further machined and finished in a subsequent machining process. During the PBF process, the dome 4 can optionally be built without support structures (i.e., stock material) under the dome 4, as shown. In other embodiments, the dome 4 can be built with support structures underneath, which are subsequently removed.

Preferably, the majority of the cage 2 can be built having a solid structure (e.g., having a 100 percent infill density). In such embodiments, one or more portions of the cage 2 can have less than 100 percent infill density, such as an infill density in a range from about 50 percent to about 99.9 percent, and can thus have less density than the solid portion(s). In such one or more portions, the infill density being less than 100 percent can be caused by such portions being porous. For example, such one or more portions of the inner surface 14 of the cage 2 can be porous, as described above, for promoting bone growth into such surface portion(s) of the cage 2. Such porous portions of the cage 2 can be rapidly manufactured according to the techniques and features more fully described in U.S. patent application Ser. No. 17/732,750, filed Apr. 29, 2022, in the name of Kavanagh, et al., the entire disclosure of which is incorporated by reference herein.

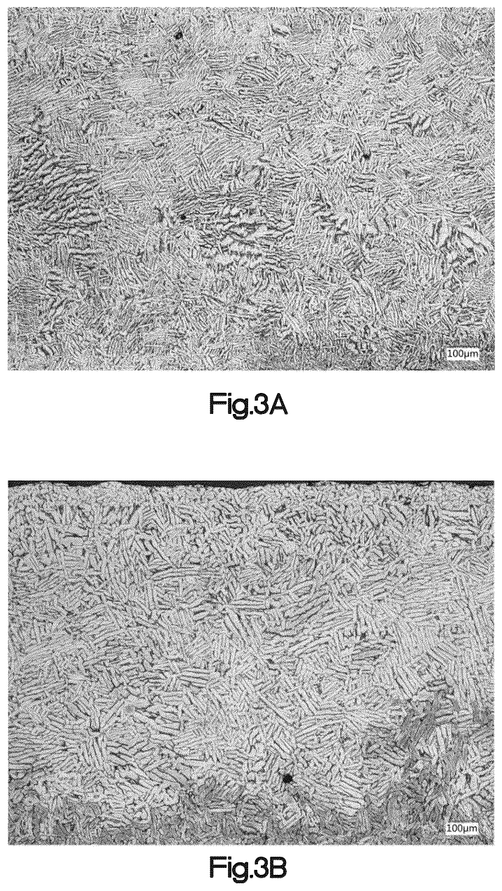

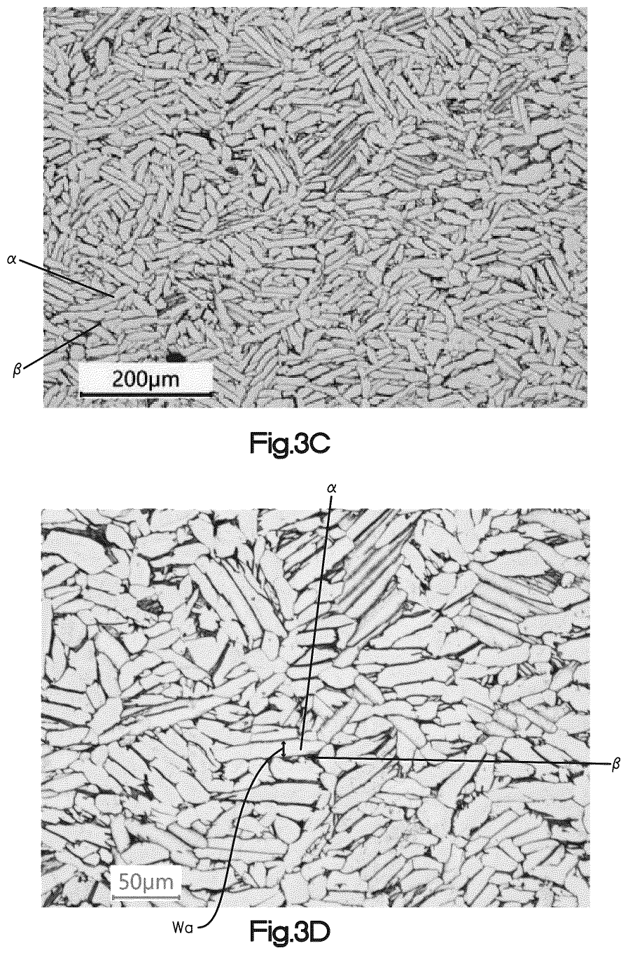

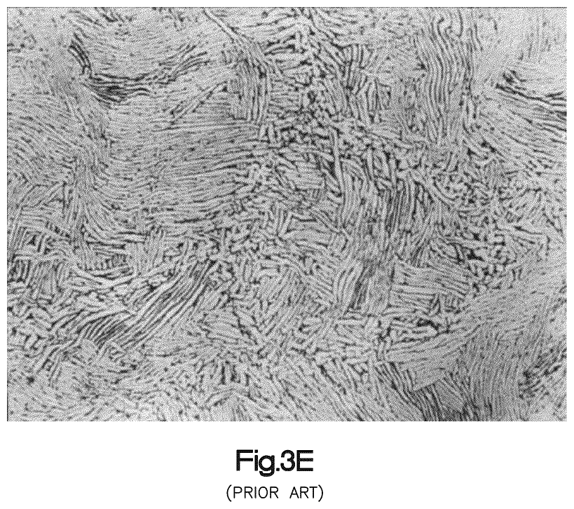

After the cage 2 is built, the cage 2 is subjected to one or more treatment processes, such as one or more heat treatment processes, for modifying the grain structure of the as-printed Ti64 implant material in a manner increasing the malleability of the cage 2, particularly for allowing a surgeon to bend portions of the cage 2 intra-operatively, without cracking, breaking, or otherwise causing mechanical failure in such portions. In particular, the Ti64 grain structure is modified from the as-printed metastable martensitic structure to lamellar alpha+beta phase with tailorable lamella size to provide the increased malleability disclosed herein. Referring now to FIGS. 3A-3D, the resulting grain structure is shown of samples of Ti64 that were printed and treated according to the embodiments herein. For example, FIGS. 3A and 3B show magnified views of exemplary grain structures of respective printed Ti64-alloy samples that were produced using Ti64 powder having the same characteristics and subjecting the printed samples to slightly different custom heat treatment processes. It can be seen that the average width of the alpha-phase lamella α in FIG. 3B are larger than those of FIG. 3A, indicating that the size of the alpha-phase lamella α can be tailored, particularly increased, which has been observed to increase malleability. Referring now to FIGS. 3C and 3D, additional magnified views show the grain structure produced using the same custom heat treatment process used to produce the grain structure shown in FIG. 3B. It should be appreciated that the alpha-phase lamella α have an increased width Wα that is larger than those of prior art Ti64 grain structures.

The treatment processes herein allow for the production of alpha-phase lamella α have an increased size, including an increased width Wα (FIG. 3D), which results in more malleable material, albeit at the expense of material strength. Cylindrical samples of printed, treated Ti64 produced according to the embodiments herein have been observed to undergo elongation in a range of 15%-23% during testing, which is a significant improvement over prior art Ti64. For comparison, FIG. 3E shows the grain structure (including lamella) of a sample of a ISO 20160 alpha+beta titanium alloy. The image shown in FIG. 3E is a micrograph A11 from ISO 20160-2006 taken at 200× magnification.

The built, treated cage 2 can be subjected to further processes, such as one or more machining, polishing, roughening, and/or coating processes as needed. For example, one or more additional holes can be formed in the cage 2, such as by drilling, milling, punching, and cutting, by way of non-limiting examples. It should be appreciated that hole(s) can be formed in the cage 2 for the purpose of increasing the cage bendability at portions adjacent the hole(s). Additionally, the built, treated cage 2 can be subjected to additional machining processes, such as wire electrical discharge machining (wire EDM), for profile cutting the cage 2, including for removing the support structures.

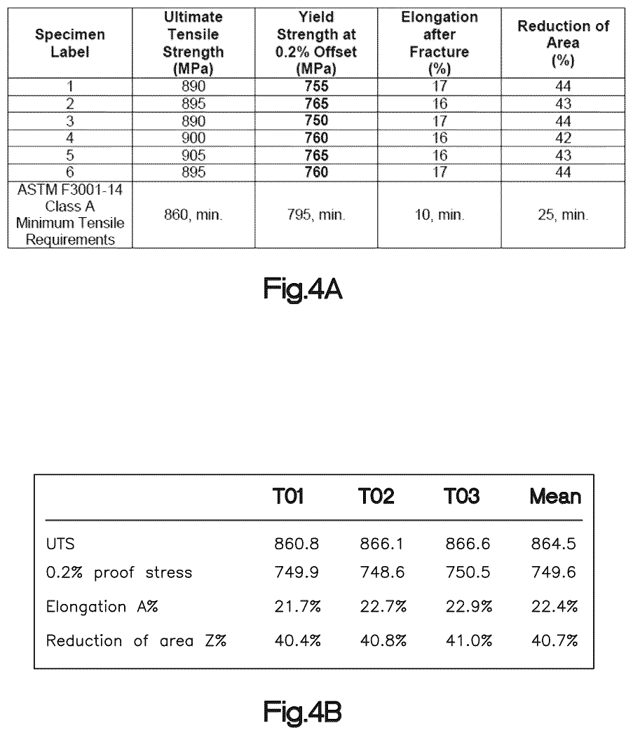

The built, treated cage 2 demonstrates material properties that have superior malleability than prior art Ti64 components of similar thickness. Referring now to FIGS. 4A-4B, two sets of tension test results are shown for printed and finished Ti64 tensile bars produced with tailored heat treatments. In FIG. 4A, six (6) specimens of printed, treated Ti64 cylinders were subjected to tensile tests at room temperature measuring the following four (4) qualities: (1) ultimate tensile strength (measured in megapascals (MPa)); (2) yield strength at 0.2% offset (also referred to as a 0.2% proof test) (measured in MPa); (3) percent elongation after fracture; and (4) percent reduction in area. For purposes of comparison, also listed are the minimum tensile requirements for these four qualities according to ASTM F3001-14. Class A. In FIG. 4B, three (3) specimens of printed, treated Ti64 cylinders were subjected to similar tensile tests, measuring various qualities, among which are the same four (4) qualities measured in FIG. 4A: (1) ultimate tensile strength (“UTS”) (measured in MPa); (2) yield strength at 0.2% offset (“0.2% proof test”) (measured in MPa); (3) percent elongation after fracture (“Elongation A %”); and (4) percent reduction in area (“Reduction of area Z %”). It should be appreciated that the samples represented in FIG. 4B were subjected to slightly different heat treatment parameters than the samples represented in FIG. 4A. The results shown in FIGS. 4A-4B demonstrate that the printed, treated Ti64 samples measured significantly high elongations, specifically from about 15% to about 23%, at mere modest sacrifices in yield strength and ultimate tensile strength. It should be noted that elongations of 15-23% represent a significant advantage over prior art 3D-printed Ti64 constructs.

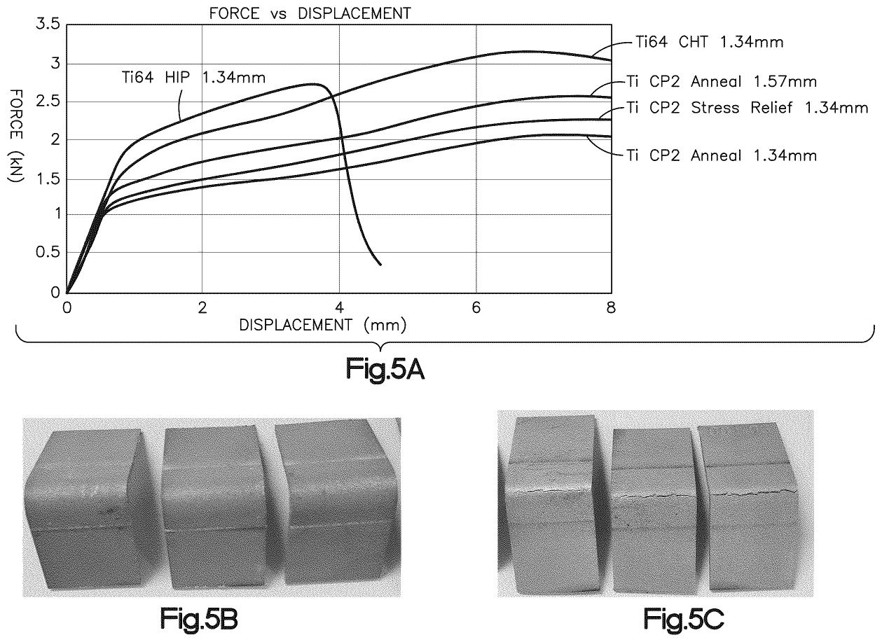

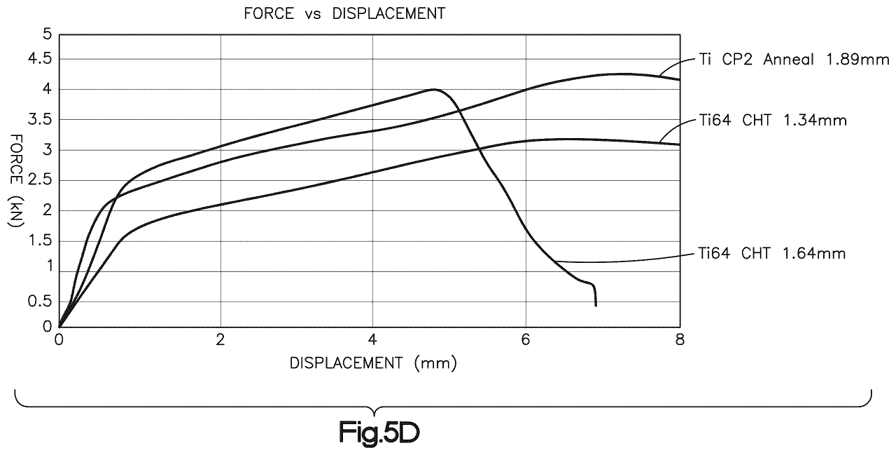

Referring now to FIGS. 5A-5C, results are shown for three-point bending tests performed on five different samples of plate-like test coupons: (1) “Ti CP2 Anneal 1.34 mm”—annealed Commercially Pure (CP) Grade 2 titanium having a thickness of 1.34 mm; (2) “Ti CP2 Stress Relief 1.34 mm”—stress relieved Commercially Pure (CP) Grade 2 titanium having a thickness of 1.34 mm; (3) “Ti CP2 Anneal 1.57 mm”—annealed Commercially Pure (CP) Grade 2 titanium having a thickness of 1.57 mm; (4) “Ti64 CHT 1.34 mm”—Ti64 alloy, heat treated (custom heat treatment (CHT)), having a thickness of 1.34 mm; and (5) “Ti64 HIP 1.34 mm”—Ti64 alloy, heat-treated via Hot Isostatic Pressing (HIP), having a thickness of 1.34 mm. The test coupons of samples (1)-(5) were rapidly manufactured using a laser-fusion PBF process and an approximate length of 50 mm and a width of 20 mm. Each sample was subject to a bending displacement target of 8 mm, which would be equivalent to a bend angle superior to 90 degrees using a test setup consistent with the requirements of ASTM E290, and the displacement versus applied force was charted. Furthermore, the bend tests were performed by bending the test coupons about a mandrel having a 5-mm diameter.

As shown in FIG. 5A, sample (4) (Ti64 CHT 1.34 mm) was able to bend to the full test displacement (equivalent to a minimum bend angle of 90 degrees), with a maximum applied force of about 3.25 kN, which is satisfactory for a cage 2 (particularly, a flange thereof) having the same thickness to be bent intra-operatively by a surgeon, such as for conforming the shape of the cage 2 to associated patient anatomy. These results compare favorably to the samples of commercially pure titanium, particularly considering the increased strength of Ti64 relative to commercially pure titanium. As shown in FIG. 5B, three test subjects of the sample (4) coupons show a clean bend and having maintained structural integrity (meaning that the samples were devoid of any indications of mechanical failure, such as cracks, delamination, or other modes of failure). As also demonstrated in these tests, the Hot Isostatic Pressing (HIP) Ti64 sample experienced cracking failure at approximately 3.75 mm displacement (applied force of about 2.75 kN), as shown in FIG. 5A, which cracks can be seen in FIG. 5C. These results demonstrate that implants constructed of Ti64 and custom heat treated have superior bending properties to those constructed of HIP Ti64.

Referring now to FIG. 5D, further bending tests indicate that cage thickness is a significant factor for bendability. The results shown in FIG. 5D are based on similar three-point bending tests discussed above with reference to FIGS. 5A-5C, performed on three different test coupons: (6) “Ti64 CHT 1.34 mm” - Ti64 alloy, heat treated, having a thickness of 1.34 mm (the same sample parameters as sample (4) shown in FIG. 5A); (7) “Ti64 CHT 1.64 mm” - Ti64 alloy, heat treated, having a thickness of 1.64 mm; and (8) “Ti CP2 Anneal 1.89 mm”—annealed Commercially Pure (CP) Grade 2 titanium having a thickness of 1.89 mm. These results demonstrate that increasing the thickness of the Ti 64, heat treated sample from 1.34 mm to 1.64 mm (an increase of 0.3 mm) may lead to failure at about 5 mm displacement (and 4.0 kN of bending force). These results indicate that, for acetabular cages 2 constructed of this material (Ti64, heat treated), portions of the cage 2 that are adapted for bending (e.g., the iliac flange 6 and/or the optional ischial flange 10) should be formed at a thickness less than 1.64 mm and preferably closer to 1.5 mm.

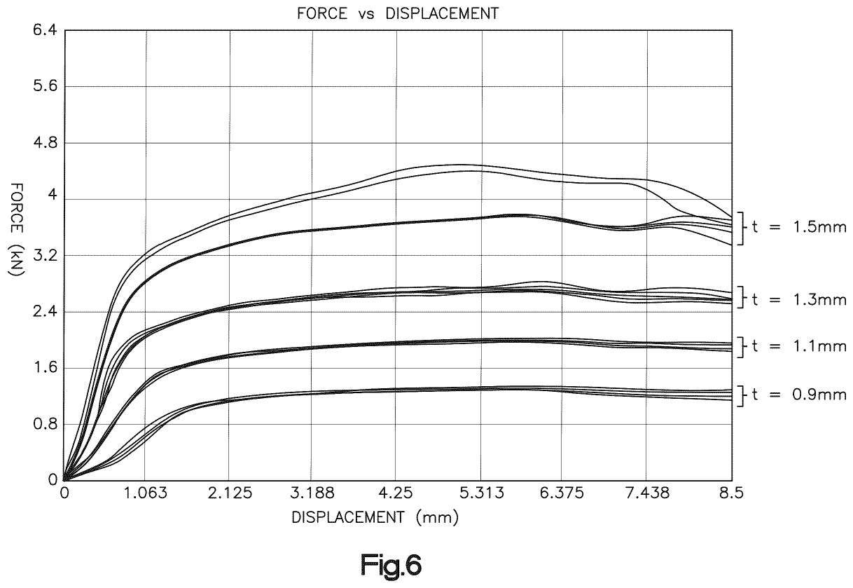

Referring now to FIG. 6, yet further bending tests indicate that implant thicknesses up to about 1.5 mm can be bent to a target test displacement of about 8 mm (equivalent to a bend angle superior to 90 degrees using a test setup consistent with the requirements of ASTM E290) without failing. In these bending tests, four (4) sample groups of test coupons having thicknesses of 0.9 mm, 1.1 mm, 1.3 mm, and 1.5 mm, respectively, were subjected to three-point bending tests similar to those discussed above with reference to FIGS. 5A-5D. Each sample group shown in FIG. 6 included eight (8) test coupons of the respective thickness. As shown, each of these sample groups was able to achieve the target test displacement (8 mm, 90+degrees) without failure.

As mentioned above, the bendable Ti64 acetabular cages 2 disclosed herein provide the strength and other associated benefits of Ti64 while also allowing a surgeon to bend portions of the cage 2 intra-operatively as needed to correspond to patient anatomy. For example, during a hip repair surgery, such as a surgery for treating an acetabular defect, such as a Type 2A, Type 2B, Type 2C, Type 3A, or Type 3B acetabular defect, the surgeon can expose and prepare the acetabulum for receiving an acetabular cup 12, such as by removing soft tissue from the acetabulum and surrounding area and optionally reaming the acetabulum. With the acetabulum prepared, the surgeon can insert the cup 12 therein with the cup holes at the desired orientation. Subsequently, the surgeon can move the acetabular cage 2 into position adjacent the cup 12, particularly by bringing the dome 4 of the cage 2 into contact with, or at least close proximity to, the concave, exposed surface of the cup 12 so that at least one of the holes 26b in the dome 4 align with at least one corresponding hole in the cup 12. With the cage 2 in such position, the surgeon can bend and shape the iliac flange 6 so as to have a contour that approximates the contour of the portion of the ilium to which the iliac flange 6 will contact.

It should be appreciated that the surgeon need not maintain the cage 2 in contact with or close proximity to the cup 2 while bending the iliac flange 6 to the desired shape. The surgeon can elect to bring the cage 2 into such position to visually reference the iliac flange 6 with respect to the corresponding portion of the ilium and then bring the cage 2 into a more comfortable position for performing the bending. This bending and shaping process can also be iterative, whereby the physician can bring the cage 12 into contact with and/or close proximity to the cup 12 to visually reference the iliac flange 6 with respect to the corresponding portion of the ilium, can then bring the cage 2 to a more comfortable position for performing the bending, and can return the cage 2 to contact with or close proximity to the cup to further visually reference the iliac flange 6 relative to the corresponding portion of the ilium, repeating these steps until a satisfactory contour and shape is implanted to the iliac flange 6.

Additionally, if the selected cage 2 includes an ischial flange 10, the surgeon can bend and shape the ischial flange 10 so as to have a contour that approximates the contour of the portion of the ischium to which the ischial flange 10 will contact. It should be appreciated that the surgeon can employ similar steps for shaping the ischial flange 10 as those described above for shaping the iliac flange 6. To affix the cage 2 and the cup 12 to the acetabulum, the surgeon can insert locking members, such as bone screws, through one or more of the aligned holes in the dome 4 and cup 12. The surgeon will also insert one or more additional locking members, such as bone screws, through one or more holes 26a in the iliac flange 6 and into underlying bone to affix the iliac flange to the corresponding portion of the ilium. If the selected cage 2 includes an ischial flange 10, the surgeon can also insert one or more additional locking members, such as bone screws, through one or more holes 26a in the ischial flange 10 and into underlying bone to affix the ischial flange 10 to the corresponding portion of the ischium. Additionally or alternatively, when the selected cage 2 includes an ischial flange 10, the surgeon can insert a portion and up to an entirety of the ischial flange 10 into the ischium, such as by drilling or otherwise forming a slot or opening in the ischium and inserting the ischial flange 10 therein. It should be appreciated that the surgeon can elect to affix a certain portion of the cage 2 to underlying bone, such as the dome 4, and shape a separate portion of the cage 2, such as the iliac flange 6 and/or the ischial flange 10, to the corresponding bone.

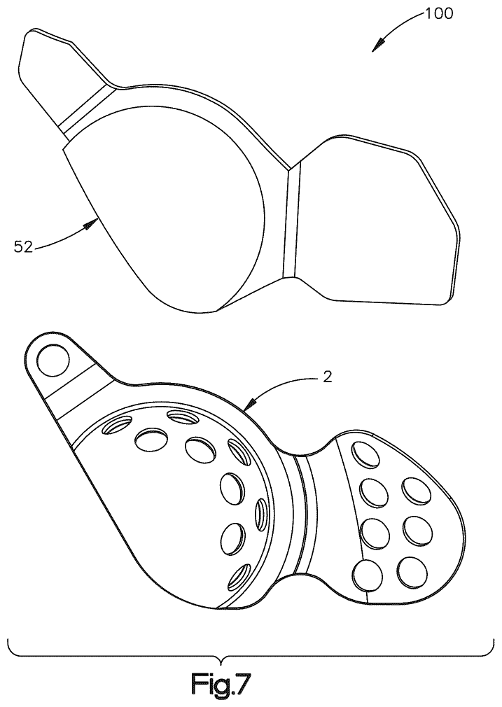

Referring now to FIG. 7, a trial system 100 for shaping an acetabular cage 2 can include a trial member 52, which is configured to be manipulated, bent, or otherwise shaped in substantially conformal fashion with associated anatomy for assisting a surgeon in bending the cage 2 to a similar shape. The trial member 52 is preferably configured to transition from a neutral state to a first state, in which the trial member 52 can be readily bent so as to match the contour of underlying anatomy. The trial member 52 is also preferably configured to transition from the first state to a second state, in which the trial member 52 substantially retains its bent/contoured shape. In this manner, a surgeon can employ the trial member 52 intra-operatively to assist in the patient-specific bending and shaping of an acetabular cage 2. For example, the trial member 52 can be employed in its first state to conform to an acetabulum (or a cup 12 disposed therein) and adjacent portions of the ilium and optionally the ischium. In particular, the surgeon can bend the trial member 52, while in its first state, to form fit around the acetabulum and adjacent portions of the ilium and optionally the ischium to which cage 2 affixation is desired. After the trial member 52 has the form fit shape, the surgeon can allow the trial member 52 to transition to the second state. After the trial member 52 is in the second state, the surgeon can remove the trial member 52 from the underlying anatomy, and employ the trial member 52 as a visual reference for bending corresponding portions of the acetabular cage 2 to the desired shape. After the cage 2 is in the desired shape, as referenced to the trial member 52, the cage 2 can be placed against the associated anatomy (and/or an associated cup 12) and affixed thereto. It should be appreciated that the cage 2 bending and shaping processes using the trial member 52 can be performed iteratively, whereby the surgeon can bend a portion of the cage 2 based on visual reference to the associated portion of the trial member 52, and place the bent portion of the cage 2 in contact with, or close proximity to, the associated anatomy to visually reference the cage 2 shape against the associated anatomy, and subsequently move the cage 2 away from the patient anatomy for further visual reference with the trial member 52 and subsequent bending.

The trial member 52 can be constructed of a material that can be activated to transition from the first state to the second state on command and within a period of time suitable for intra-operative bending and shaping. Preferably, the material for the trial member 52 is also suitable for rapid manufacturing. Non-limiting examples of such materials that are suitable for rapid manufacturing and are also activatable into the first or second state include polymeric materials, such as polylactic acid (PLA), poly(lactic-co-glycolic acid) (PLGA), and the like, and metals, such as titanium (e.g., commercially pure titanium), titanium alloys, stainless steel, and other medical-grade metals. PLA, PLGA, and similar polymeric materials can be 3D-printed in a neutral state, and can be activated into the first (malleable) state by heating the material beyond its glass transition temperature. While in this first state, the surgeon can bend, shape, or otherwise form fit the trial member 52 to the corresponding anatomy. Afterward, the material will set (i.e., harden) in the shaped form, thereby providing a hardened, shaped trial member 52 that the surgeon can employ as a visual and tactile reference when bending and shaping the cage 2. In other embodiments, the material for the trial member 52 can be activated into the first or second state by application of UV light or other energies. In yet other embodiments, the material for the trial member 52 need not be 3D-printable but can instead be formed from stock sheet material that can be cut, trimmed, or otherwise shaped similar to the geometry of the cage 2. In other embodiments, the trial member 52 can be formed from a stock sheet of PLA, PLGA, or similar material, which is placed in a rapidly manufactured (e.g., 3D-printed) mould and subsequently molded therein to a shape replicating the shape of the cage 2. In some embodiments, the material of the trial member 52 can be substantially transparent when in the neutral and/or first state. In yet other embodiments, the stock sheet can be constructed of titanium, a titanium alloy, stainless steel, or another medical-grade metal. It should also be appreciated that the trial member 52 need not have fixation holes 26a, b formed therein. Materials such as PLA and PLGA are beneficial candidates for constituent materials of the trial member 52 because they can be 3D-printed into the shape of the cage 2 or into a mould for vacuum forming the trial member 52, can be provided in sheet form, and are substantially transparent in the neutral state. It should be appreciated that the cage 2 and one or more trial members 52 can be included together in a kit, in which each trial member 52 can be a single-use trial member.

It should be appreciated that the various parameters of the implants described above, and the related techniques for constructing, treating, machining, bending, and shaping these implants are provided as exemplary features for adapting the implants to substantially conform to patient anatomy. These parameters can be adjusted as needed without departing from the scope of the present disclosure.

It should further be appreciated when a numerical preposition (e.g., “first”, “second”, “third”) is used herein with reference to an element, component, dimension, or a feature thereof (e.g., “first” flange, “second” flange, “first” state, “second” state), such numerical preposition is used to distinguish said element, component, dimension, and/or feature from another such element, component, dimension and/or feature, and is not to be limited to the specific numerical preposition used in that instance. For example, a “first” member can also be referred to as a “second” member in a different context without departing from the scope of the present disclosure, so long as said elements, components, dimensions and/or features remain properly distinguished in the context in which the numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An intraoperatively-bendable acetabular cage, comprising:
a dome for insertion within an acetabulum; and
a flange for affixation to a portion of an ilium, the flange and the dome being monolithic with each other,
wherein the dome and the flange are constructed of fused particles of a Ti64 alloy exhibiting an alpha+beta phase lamellar grain structure having lamella of a greater width than lamella of fused particles of the Ti64 alloy following particle fusion.

2. The intraoperatively-bendable acetabular cage of claim 1, wherein at least a portion of the flange is bendable to a bend angle of at least about 90 degrees about a bend axis while maintaining structural integrity.

3. The intraoperatively-bendable acetabular cage of claim 2, wherein the at least a portion of the flange is bendable to a bend angle of at least about 90 degrees about a bend axis with a force of no more than 3.25 kN.

4. The acetabular cage of claim 2, wherein the at least a portion of the flange has a thickness in a range of about 0.9 mm to about 1.5 mm.

5. The intraoperatively-bendable acetabular cage of claim 1, wherein the flange has a thickness of less than 1.64 mm.

6. The intraoperatively-bendable acetabular cage of claim 1, wherein the cage has an overall infill density of at least 95 percent.

7. The intraoperatively-bendable acetabular cage of claim 1, wherein the flange defines a first surface and a second surface opposite the first surface, the flange defines at least one hole that extends between the first and second surfaces and is configured for receiving a bone fixation member for affixing the flange to bone, the at least one hole extends along a central hole axis that is oriented at an angle relative to a transverse axis that is substantially orthogonal to the first and second surfaces at a location adjacent the at least one hole.

8. The intraoperatively-bendable cage of claim 1, further comprising a further flange that extends from the dome at a location generally opposite the flange.

9. The intraoperatively-bendable cage of claim 7, wherein the further flange is configured for affixation to the ischium of the pelvis.

10. The intraoperatively-bendable cage of claim 1, wherein the intraoperatively-bendable cage is sufficiently malleable to allow a user surgeon to bend and shape portions thereof by hand so as to conform to the shape of adjacent patient anatomy.

11. The intraoperatively-bendable cage of claim 10, wherein the intraoperatively-bendable cage is sufficiently malleable to allow a user surgeon to bend and shape portions thereof by hand without cracking, breaking, or otherwise causing mechanical failure in such portions.

12. The intraoperatively-bendable cage of claim 1, wherein the intraoperatively-bendable cage defines a thickness measured between corresponding portions of inner and outer surfaces along a direction orthogonal thereto, the thickness in a range of about 0.5 mm to about 3.0 mm.

13. The intraoperatively-bendable cage of claim 12, wherein the thickness is in a range of about 0.9 mm to about 2.2 mm.

14. The intraoperatively-bendable cage of claim 13, wherein the thickness is in a range of about 0.9 mm to about 1.5 mm.

15. The intraoperatively-bendable cage of claim 12, wherein one or more portions of the intraoperatively-bendable cage have a reduced thickness relative to various other portions.

16. The intraoperatively-bendable cage of claim 15 wherein the one or more portions of the intraoperatively-bendable cage are configured to facilitate bending and shaping along the one or more portions.

17. The intraoperatively-bendable cage of claim 1, wherein the flange defines a bend at a portion thereof proximate the dome.

18. The intraoperatively-bendable cage of claim 17, wherein the bend is characterized as an S-type bend.

19. The intraoperatively-bendable cage of claim 1, wherein the iliac flange defines a neck adjacent the dome.

20. A method of preparing an intraoperatively-bendable acetabular cage for implantation, the method comprising:
with an intraoperatively-bendable acetabular cage that comprises: a dome for insertion within an acetabulum; and a flange for affixation to a portion of an ilium, the flange and the dome being monolithic with each other, wherein the dome and the flange are constructed of fused particles of a Ti64 alloy exhibiting an alpha+beta phase lamellar grain structure having lamella of a greater width than lamella of fused particles of the Ti64 alloy following particle fusion,
bending a portion of the intraoperatively-bendable acetabular cage to a bend angle of at least about 50-degrees about a bend axis, the portion maintaining structural integrity during and after the bending.
